SEQUENCE LISTING (4 sequences)

SEQ ID NO: 1 - DNA, 23 bp, Artificial
  primer for PCR
gcggcaggcctaacacatgcaag

SEQ ID NO: 2 - DNA, 22 bp, Artificial
  primer for PCR
atccaaccgcaggttcccctac

SEQ ID NO: 3 - DNA, 25 bp, Artificial
  primer for PCR
cggacctcataaagtgcgtcgtagt

SEQ ID NO: 4 - DNA, 25 bp, Artificial
  primer for PCR
cgtacgatcgatgtagctagcatgc

(19) United States
(12) Patent Application Publication
(10) Pub. No.: US 2006/0029959 A1
(43) Pub. Date: Feb. 9, 2006

(54) Detection method of detecting nucleic acid amplification reaction product and kit for detecting nucleic acid

(75) Inventors: Tadashi Okamoto, Yokohama-shi (JP); Ikumasa Ikeda, Miura-gun (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/194,688

(22) Filed: Aug. 2, 2005

(30) Foreign Application Priority Data
Aug. 4, 2004 (JP) 2004-228344

(51) Int. Cl. C12Q 1/68

(52) U.S. Cl. 435/6

(57) ABSTRACT

Provided is a strand separation method of a double strand DNA as an amplification product, which facilitates the strand separation of each strand composing a double strand DNA after nucleic acid amplification reaction and allows the acquisition and detection of a target nucleic acid with higher efficiency through the use of PCR. A primer bound with a fine particle for capture is used to perform PCR, and each strand forming a double strand DNA as a resulting amplification product is separated and collected using the fine particle bound with the primer.

### BACKGROUND OF THE INVENTION

1. Field of the Invention

The present invention relates to a detection method of detecting a nucleic acid amplification reaction product and a kit for detecting a nucleic acid.

2. Related Background Art

The strand extension/amplification of nucleic acids using primers, so-called PCR (polymerase chain reaction), is utilized in the separation and purification of nucleic acids from organisms, the detection of target nucleic acids in a variety of nucleic acid samples, genetic testing, and so on, as a method useful for amplifying nucleic acid fragments having nucleotide sequences of interest by strand extension reaction to obtain the nucleic acid fragments in amounts sufficient for subsequent purification and detection even if nucleic acid samples initially used are in trace amounts.

Nucleic acids are amplified as double strands in PCR. This double strand nucleic acid is dissociated into single strands, both or either of which are (is) used, depending on the applications of an amplification product.

Fluorescent labeling for modifying an amplified nucleic acid with fluorochrome or the like, a method of chemiluminescence detection, and so on, have been proposed as techniques for adding a detectable label to the obtained amplification product.

Methods for modification include a method of using a primer attached to a label and a method of labeling a monomer incorporated during PCR.

However, for all of these methods, a large amount of an amplified product is required to achieve an amplification level detectable by a technique typically used, and much time is spent in this amplification treatment.

In general, the target nucleic acid is fluorescently labeled with a fluorescently-labeled primer or a fluorescently-labeled nucleotide monomer labeled with a fluorescent material when subjected to amplification reaction, and the target nucleic acid thus labeled is then detected by solid phase hybridization reaction such as a DNA microarray and a fluorescence method. The fluorescence method can be said to be a relatively convenient method and to have sufficiently high sensitivity in practical use and however, requires a special detection apparatus in an attempt to detect, for example, one target nucleic acid molecule. In this sense, means for detecting hybridization more easily with higher sensitivity has been desired.

### SUMMARY OF THE INVENTION

An object of the present invention is to provide convenient and high-sensitivity detection of an amplified target nucleic acid.

The present invention has been completed in light of the above-described problems of conventional arts and includes each of methods below.

A method of detecting a target nucleic acid according to the present invention is
a detection method of detecting a target nucleic acid using plural nucleic acid probes on the surface of a substrate, comprising the steps of:
providing a target nucleic acid or a nucleic acid as an amplification reaction product thereof bound with a fine particle;
hybridizing the nucleic acid bound with the fine particle to said immobilized nucleic acid probes; and
detecting the fine particle on the substrate.

It is preferred that the step of providing a nucleic acid primer bound with a fine particle; and subjecting a sample containing the target nucleic acid to strand extension reaction with the primer to obtain a reaction product having an extended strand.

It is also preferred that the step of obtaining a reaction product should be the step of performing PCR (polymerase chain reaction).

The step of obtaining a reaction product may be the step of performing PCR using a primer set containing a first primer used as the primer bound with a fine particle and a second primer that is not bound with a fine particle.

The step of obtaining a reaction product may employ plural sets of primers.

It is preferred that the method of detecting a target nucleic acid should further comprise the step of performing strand separation for separating one single strand DNA having the primer bound with the fine particle as the amplification reaction product, from another single strand DNA by capturing the fine particle of the primer.

The strand separation may be performed by centrifugation, or may by performed magnetically with a magnetic fine particle.

It is preferred that the first primer should be designed for the extension of a strand with a target sequence, and that a hybrid formed by the hybridization should be detected by detecting the fine particle.

Preferably, the first primer is designed for the extension of a strand without a target sequence and the second primer is designed for the extension of a strand with a target sequence.

It is preferred that the primer bound with the fine particle should be a primer for linear amplification, and that the step of obtaining a reaction product should be the step of subjecting a nucleic acid group containing the target nucleic acid to linear amplification reaction with the primer to obtain an amplification reaction product.

Plural primers may simultaneously be used. In this case as well, the fine particle is preferably a magnetic fine particle.

The detection may be performed using scanning probe microscopy (SPM). It is particularly preferred that the SPM is magnetic force microscopy (MFM).

The present invention also provides a kit for detecting a target nucleic acid.

That is, a kit for PCR reaction to detect a target nucleic acid according to the present invention, comprising:
plural nucleic acid probes immobilized on the surface of a substrate; and
a primer bound with a fine particle for PCR.

The fine particle is preferably a magnetic fine particle.

The present invention allows convenient and high-sensitivity detection in combination with detection using a nucleic acid probe chip. Moreover, the use of the primer bound with a fine particle of the present invention facilitates strand separation after nucleic acid amplification reaction and allows the more convenient detection of a nucleic acid with higher sensitivity.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a diagram showing the outline of a hybridization apparatus capable of application of magnetic force;

FIG. 2 is a diagram showing one example of a relationship between a sample and a primer;

FIG. 3 is a diagram showing the outline of one example of a strand separation method;

FIG. 4 consisting of FIGS. 4A and 4B is a diagram showing the outline of one example of a strand separation method;

FIG. 5 consisting of FIGS. 5A and 5B is a diagram showing the outline of one example of a strand separation method; and

FIG. 6 is a diagram showing the outline of one example of a strand separation method.

### DETAILED DESCRIPTION OF THE PREFERRED EMBODIMENTS

Preferred embodiments of the present invention will now be described in detail in accordance with the accompanying drawings.

(Method of Detecting Target Nucleic Acid)

A detection method of detecting a target nucleic acid according to the present invention is a method of detecting a target single strand DNA using plural nucleic-acid probes immobilized on the surface of a substrate, which is a method of detecting a target nucleic acid using plural nucleic acid probes immobilized on the surface of a substrate, comprising the steps of:
providing a target nucleic acid or a nucleic acid as an amplification reaction product thereof bound with a fine particle;
hybridizing the nucleic acid bound with the fine particle to plural nucleic acid probes immobilized on the surface of a substrate; and
detecting the fine particle on the substrate.

To be more specific, the method comprises the steps of:
providing a primer bound with a fine particle formed by a primer bound with a fine particle;
subjecting a sample containing the target nucleic acid to strand extension reaction with the primer bound with a fine particle to obtain a reaction product having an extended strand;
hybridizing the reaction product to the plural nucleic acid probes immobilized on the surface of a substrate; and
detecting the fine particle bound with the reaction product on the substrate.

In this method, the amplified double strand nucleic acid can be detected with the probe without the need to separate its strands, and the use of the fine particle in this detection allows the convenient and high-sensitivity detection of the nucleic acid.

A more detailed aspect of the method of detecting a target nucleic acid of the present invention is a method of detecting a (single strand) nucleic acid separated by a strand separation method using a so-called nucleic acid probe chip that has the surface bound with plural nucleic acid probes, comprising, when a nucleic acid group containing the target nucleic acid is subjected to amplification reaction using two types of primers, binding one of the primers to a fine particle and separating a double strand amplification reaction product after amplification reaction into single strands by use of the fine particle to detect the separated single strand target nucleic acid by using hybridization with the nucleic acid probe chip.

In the present invention, it is possible to use plural primer sets. The use of the probe chip in combination with the primer sets is more preferable because plural amplification products can simultaneously be detected.

The method of detecting the amplification product using the probe chip includes a method in which, of two types of primers, a primer designed for the extension of strand having a target sequence is bound with the fine particle and the hybridization is detected by detecting the fine particle. This method allows detection with higher sensitivity than those of conventional methods, as described below.

It is preferred that PCR amplification reaction should be performed as with, for example, PCR conducted in amplification cycles repeated 20 to 30 times using two types of primers. However, in some cases, when a nucleic acid group containing the target nucleic acid is subjected to (linear) amplification reaction using one type of primer, the primer may be bound with the fine particle, and the single strand target nucleic acid after amplification reaction may be detected using hybridization with the nucleic acid probe chip. In either of cases where two types of primers are used or where one type of primer is used, the amplification cycle may be repeated once to plural times when high-sensitivity detection can be attained.

In the detecting method of the present invention, the use of a magnetic fine particle as the fine particle permits easy strand separation and further allows the stirring of a reaction solution by the application of a magnetic filed form the outside source and the promotion of reaction by placing the target nucleic acid in the proximity of the nucleic acid probe, when hybridization reaction is performed.

In the detecting method of the present invention, any means for detecting a subject to be detected may be used as long as the means is capable of detecting the fine particle. When both of two types of primers are bound with fine particles, it is preferred that the means is constructed so that one of the fine particles can selectively be detected.

The shape and size of the fine particle used in each method of the present invention should not be particularly limited and is appropriated selected depending on the material, composition and production method of the fine particle, a binding method of the primer, the rates of amplification reaction and hybridization reaction, the number of a primer bound per fine particle, a detection method, detection sensitivity, and so on. Taking each of the requirements into consideration, atypical and typical fine particles each having a size of 0.1 to 1000 nm corresponding to its particle diameter as well as rodlike, needle-shaped and fragmentary fine particles each having at least two-dimensional size (of length, width and height) of 0.1 to 1000 nm can preferable be employed as the fine particle.

Means that can be used for detecting the fine particle having such a size includes, without limitations, light microscopy, electron microscopy, surface analysis methods capable of two-dimensional imaging such as time-of-flight secondary ion mass spectrometry (TOF-SIMS), X-ray photoelectron spectroscopy (XPS) and Auger electron spectroscopy (AES), and a variety of scanning probe microscopies (SPM) capable of detecting three-dimensional shapes relatively conveniently with high sensitivity. Especially when the fine particle is a magnetic fine particle, the use of magnetic force microscopy (MFM), one type of SPM, allows rapid detection and sometimes allows detection with precision and high reliability because magnetic information is acquired along with shape information and therefore, in some cases, the fine particle can be measured in non-contact mode. When the magnetic particle is used, more efficient amplification reaction is made possible by stirring a reaction solution with a magnetic field applied to the reaction solution during PCR or by increasing chances for contact of the nucleic acid to be amplified with the primer.

Especially when the detecting method with high sensitivity as described above is used, an amplification product even with a low concentration is sometimes detected satisfactorily. In such a case, strand separation is not needed in particular. One embodiment of the present invention encompasses a method of detecting an amplification product labeled with a particle by SPM, MFM, or the like without performing strand separation.

Any of methods for producing the fine particle used in the present invention and for binding the nucleic acid primer used in the present invention may be utilized as long as the function of the present invention is achieved. For example, a method described in Japanese Patent Application Laid-Open No. 2004-065199 and 2000-102400 that will be described below may be utilized.

(Strand Separation Method)

A nucleic acid after amplification reaction by PCR is detected, in some cases, using reaction that forms a hybrid (hybridization) with a probe nucleic acid having a base sequence complementary to a particular portion of the base sequence of a nucleic acid desired to be detected (target nucleic acid), while in general, the nucleic acid after amplification reaction is a nucleic acid having two complementary strands. However, the acquisition of this nucleic acid amplified a double strand sometimes reduces the efficiency of subsequent steps. An example thereof is a case where, when double strand nucleic acids containing the target nucleic acid after amplification are separated into single strands and placed along with probes under hybridization conditions, the formation of a hybrid between the probe and the target nucleic acid occurs competitively with the formation of a hybrid between two complementary strands of the nucleic acid after amplification and is thereby inhibited, with the result that desired detection is not performed efficiently. In such a case, taking PCR as an example, a method has been known, by which one of two types of primers is attached to biotin and an amplification product after PCR is passed through an avidin column to thereby perform strand separation. However, this method presents problems such as complicated operation and the condensation of the (single strand) target nucleic acid eluted from the column, which is sometimes required. Thus, more efficient strand separation method has been desired.

A strand separation method according to the present invention utilizes a primer that is bound with a fine particle so that the fine particle for capture available in strand separation is introduced into one strand of an amplified double strand DNA, in order to accurately separate the respective strands of the amplified (including one extension procedure) double strand DNA and to efficiently separate and collect a desired single strand DNA from these separated strands. Amplification reaction performed with a primer bound with a particle is described in Japanese Patent Application Laid-Open No. 2000-102400. However, in the amplification reaction used therein, a portion of the primer is bound with a magnetic fine particle which is in turn moved by a magnetic field allowed to act on a reaction solution during amplification reaction, thereby stirring the reaction solution to improve the efficiency of the reaction. Thus, the application does not disclose a method of performing strand separation by binding one of primers to a fine particle as described in the present invention. Alternatively, Japanese Patent Application Laid-Open No. 2004-065199 discloses a method of performing amplification reaction by binding a primer to a fine particle for improvement in reaction efficiency and detection of much practical use having simple operation. Although the binding of one of primers to a fine particle is also described in the published application, no mention is made of strand separation that is a requirement in the present invention.

Japanese Patent Publication No. H10-510158 discloses amplification with a primer immobilized on a magnetic fine particle, which uses magnetic cycle reaction for the purpose of achieving amplification reaction with faster throughput and more excellent accuracy. However, the use of the magnetic fine particle as a label in detection is not disclosed by any means.

The strand separation method according to the present invention permits efficient hybridization. In this case, plural sets of primers as well as plural nucleic acids or nucleic acid sites to be detected may be used for one reaction field.

According to the strand separation method of the present invention, the fine particle is bound with one strand of an amplification product. Therefore, the strand separation may be performed by, for example, centrifugation and, when the fine particle is a magnetic fine particle, the strand separation may be magnetically performed. Thus, convenient strand separation having easy operation is made possible.

Specifically, the strand separation method according to the present invention comprises the following steps:
(a) providing a primer set for amplification consisting of a first primer and a second primer, wherein the first primer is bound with a fine particle for capture;
(b) using the primer set to obtain a double strand DNA as an amplification reaction product; and
(c) performing strand separation for separating a single strand DNA having the first primer that is one strand composing the double strand DNA as the amplification reaction product, from a single strand DNA having the second primer that is another strand by capturing the fine particle of the first primer.

Here, a nucleic acid sample as a subject to be amplified in PCR for obtaining the double strand DNA as the amplification reaction product of can be utilized in a form of a single strand DNA having a base sequence to be amplified or a double strand nucleic acid formed between the single strand DNA and its complementary sequence, in the strand separation method of the present invention.

When a target nucleic acid and the other nucleic acids (which may be single-stranded or double-stranded) are mixed in a sample and the direct amplification of the sample by PCR results in no improvement in reaction efficiency, a stand separation method comprising the following steps can preferably be utilized:
(a) providing a sample containing a target single strand DNA;
(b) providing a primer set consisting of a first primer and a second primer, wherein the first primer is bound with a fine particle for capture;
(c) hybridizing the first primer with the target single strand DNA in the sample to obtain a hybrid;
(d) capturing the hybrid using the fine particle of the hybrid;
(e) performing amplification reaction using the captured hybrid and the second primer to obtain an amplification reaction product; and
(f) performing strand separation for separating a single strand DNA having the first primer that is one strand composing a double strand DNA as the amplification reaction product, from a single strand DNA having the second primer which is another strand In this method, the target nucleic acid can be used in amplification reaction after the nucleic acid to be amplified is efficiently separated from foreign nucleic acids in the sample using the primer bound with the fine particle. In addition, each strand (single strand) of the double strand DNA as the amplification reaction product can efficiently be separated using the fine particle again. In this case, the target single nucleic acid may be contained in a form of a single strand in a sample or may be contained in a form of double strand in a sample. Moreover, the first primer having the fine particle may be added during amplification reaction at the step (e). That is, the nucleic acid to be amplified that has been separated from foreign nucleic acids in a sample by use of the fine particle is subjected to amplification reaction using the first and second primers, thereby allowing the separation and collection of the desired strand from the amplified double strand DNA by use of the fine particle. The timing and amount of the first and second primers added can variously be selected within the range that can attain strand separation of interest by this method. Furthermore, the separating property of the fine particle bound with the primer when the target single strand DNA is separated from a sample by hybridization may be different from that of the fine particle bound with the primer during amplification reaction.

A specific example of the present embodiment is a strand separation method of a double strand amplification reaction product where only a desired nucleic acid has been separated and amplified from a mixture solution containing plural single or double strand nucleic acids, which is performed in the following way: a fine particle is bound with one primer of two types of primers for performing amplification; the fine particle is added to the mixture solution to bind a desired nucleic acid to the primer bound with the fine particle: and the fine particle is separated from the mixture solution while being trapped. Thereafter, the fine particle is mixed with an amplification solution to perform amplification reaction, and a double strand DNA amplification reaction product after amplification reaction (including one extension procedure) is separated into (two) single strand DNAs using the fine particle. This allows the strand separation of a double strand amplification reaction product where only a desired nucleic acid has been separated and amplified.

Hereinafter, embodiments of the present invention will be described more fully with emphasis on a case in which a magnetic fine particle is used. Magnetic particles and nucleic acid primers, methods of binding magnetic particles to primers, hybridization reaction methods, and nucleic acid probe chips include, but not limited to, the followings:

The magnetic particle described in the present invention may be any particle of at least one type selected from the group consisting of a magnetic fine particle, a magnetic nanoparticle, and a fine particle coated with a magnetic substance.

A material for the magnetic particle that may be used is not particularly limited and includes nickel and iron particles, a variety of ferrites themselves such as Fe₃O₄, γ-Fe₂O₃, Co-γ-Fe₂O₃, (NiCuZn)O. (CuZn)O.Fe₂O₃, (MnZn)O.Fe₂O₃, (NiZn)O.Fe₂O₃, SrO.6Fe₂O₃, BaO.6Fe₂O₃ and Fe₃O₄ coated with SiO₂ (particle diameter: 200 A) [see Enzyme Microb. Technol., vol. 2, p2-10 (1980)], and composite particles between these ferrites and a variety of high-molecular materials (e.g., nylon, polyacrylamide and polystyrene).

A substrate material for a probe chip that may be used in the present invention includes glass, plastic, silica and metal. For these materials, it is important that raw materials that are not magnetized during the application of a magnetic field in hybridization reaction between the probe chip and a magnetic fine particle-bound target nucleic acid are chosen.

In the present invention, the binding of the (magnetic) fine particle to a primer can be attained by introducing a first functional group into the end of the primer and a second functional group capable of binding to the first functional group into the fine particle, and then binding these two functional groups. A method for binding the fine particle and the primer includes physical adsorption, ionic bond and covalent bond methods. The physical adsorption method is a method for immobilizing a fine particle and a primer by hydrophobic interaction working therebetween, in which the fine particle is merely immersed in a primer solution and thereby adsorbed to the primer. The ionic bond method utilizes a bond between a cation and an anion attracted through electrostatic force. The physical adsorption and ionic bond methods are not so much preferred in the present invention because they are reversible binding methods. In contrast, the covalent bond method can attain a bond stronger than those of other binding methods by introducing functional groups that can be covalently linked together into both fine particle and primer and reacting these two functional groups.

More concrete combinations thereof include an isocyanate, epoxy, formyl or mercapto group as the second functional group introduced into a fine particle and an amino group as the first functional group introduced into a primer. Alternatively, the second functional group introduced into a magnetic particle and the first functional group introduced into a primer may be reversed.

Further examples thereof include a maleimidyl, α,β-unsaturated carbonyl, α-halocarbonyl, alkyl halide, aziridine or disulfide group as the second functional group introduced into a fine particle and a thiol group as the first functional group introduced into a primer. Alternatively, the second functional group introduced into a fine particle and the first functional group introduced into a primer may be reversed.

A commercially-available fine particle into which a functional group is introduced may be used.

For binding a primer onto a fine particle, the primer can be immobilized on the fine particle by sequential extension and synthesis on the fine particle.

Alternatively, the fine particle may be bound with an amplification reaction product amplified with the primer. In this case, the fine particle and the amplification reaction product may be bound together by such a conventionally-known method that the primer having the first functional group as described above is used to perform amplification treatment, followed by the binding of the fine particle to the amplification product.

The binding of the target nucleic acid and the fine particle may be a bond via hybridization. In this case, as in a conventional sandwich hybridization method, a hybrid formed by hybridization between a fine particle bound with oligonucleotide and a target nucleic acid may be reacted with a nucleic acid probe chip illustrated below.

The nucleic acid probe chip used in the present invention may be any nucleic acid probe. One example of the production of the nucleic acid probe chip by spotting a nucleic acid probe solution on a substrate will be illustrated below.

A spotting solution used in the spotting of probes to a substrate includes a solution obtained by providing a solution containing 7.5% by mass of glycerin, 7.5% by mass of urea, 7.5% by mass of thiodiglycol and 1.0% by mass of acetylene alcohol (trade name: Acetylenol EH, manufactured by Kawaken Fine Chemicals) which is then added to a solution containing a single strand DNA (a TE solution (10 mM Tris-HCl (pH 8)/1 mM EDTA solution) having a single strand DNA concentration of approximately 400 mg/ml) to adjust the final concentration of the single strand DNA to 8 μM. This probe solution has a surface tension within the range of 20 to 50 dyne/cm and also has a viscosity from 1.2 to 2.0 cps (measured by an E-type viscometer manufactured by Tokyo Keiki).

Methods for spotting the spotting solution containing the probes to a substrate include, but not limited to, pin and ink-jet formats. However, any type of method may be used as long as the method can spot the spotting solution containing the probes to a substrate. In the ink-jet format, any of liquid jet apparatuses by thermal-jet and piezoelectric systems can be employed.

A method where DNA nucleic acid probes are sequentially synthesized on a substrate by photolithography, as described in U.S. Pat. No. 5,405,783, may be used.

As described above, the use of the magnetic fine particle can provide a consistent system that employs an identical magnetic primer to procedures especially from the extraction and separation of desired DNA through the amplification and detection of the DNA.

### EXAMPLES

Hereinafter, the present invention will be described in more detail with reference to Examples.

### Example 1

### Detection of E. coli Genomic DNA

(1) Extraction of E. coli Genomic DNA

At first, an E. coli reference strain was cultured according to a standard method. A 1.0-ml aliquot (OD₆₀₀=0.7) of the microorganism culture solution was collected into a 1.5-ml microtube and centrifuged (8500 rpm, 5 min., 4° C.) to recover bacterial cells. After the supernatant was discarded, the bacterial cells were supplemented with 300 μl of Enzyme Buffer (50 mM Tris-HCl (pH 8.0), 25 mM EDTA) and resuspended using a mixer. The resuspended bacterial solution was centrifuged again (8500 rpm, 5 min., 4° C.) to recover bacterial cells. After the supernatant was discarded, the recovered bacterial cells were supplemented with the following enzyme solutions and resuspended using a mixer: Lysozyme: 50 μl (20 mg/ml in Enzyme Buffer); and N-Acetylmuramidase SG: 50 μl (0.2 mg/ml in Enzyme Buffer).

Next, the bacterial solution that had been supplemented with the enzyme solutions and resuspended was left standing in an incubator at 37° C. for 30 minutes to dissolve the cell walls. Subsequently, genomic DNA was extracted using a nucleic acid purification kit (MagExtractor-Genome-, manufactured by TOYOBO).

Specifically, at first, the pretreated lysate of the microorganism was supplemented with 750 μl of a dissolving/adsorbing solution and 40 μl of magnetic beads and vigorously stirred for 10 minutes using a tube mixer (Step 1). The microtube was placed in a stand for separation (Magical Trapper) and left standing for 30 seconds to gather the magnetic particles on the wall surface of the tube, followed by the removal of the supernatant with the tube held in the stand (Step 2). Following the addition of 900 μl of a washing solution, the resulting solution was stirred for approximately 5 seconds with a mixer and resuspended (Step 3). Next, the microtube was placed in the stand for separation (Magical Trapper) and left standing for 30 seconds to gather the magnetic particles on the wall surface of the tube, followed by the removal of the supernatant with the tube held in the stand (Step 4). After Steps 3 and 4 were repeated and second washing (Step 5) was performed, the resulting solution was supplemented with 900 μl of 70% ethanol, then stirred for approximately 5 seconds with a mixer, and resuspended (Step 6). Next, the microtube was placed in the stand for separation (Magical Trapper) and left standing for 30 seconds to gather the magnetic particles on the wall surface of the tube, followed by the removal of the supernatant with the tube held in the stand (Step 7). After Steps 6 and 7 were repeated and second washing with 70% ethanol (Step 8) was performed, the recovered magnetic particles were supplemented with 100 μl of pure water and stirred for 10 minutes with a tube mixer. Subsequently, the microtube was placed in the stand for separation (Magical Trapper) and left standing for 30 seconds to gather the magnetic particles on the wall surface of the tube, and the supernatant was recovered into a new tube with the tube held in the stand. The recovered genomic DNA of the E. coli was subjected to agarose electrophoresis and absorbance measurement at 260/280 nm according to a standard method to examine its quality (the content of low-molecular nucleic acids and the degree of degradation) and recovered amount. In the present Example, approximately 9 μg of the genomic DNA was recovered, and the degradation of the genomic DNA and the presence of rRNA were not observed. The recovered genomic DNA was dissolved in a TE buffer to bring the final concentration to 5 ng/μl, and used in Examples below.

(2) Preparation of PCR Primers

Following oligonucleotides were prepared as primers for amplifying, by PCR, the 16S rRNA gene region of the E. coli genome prepared in (1):

| | |
|---|---|
| Forward primer: | 5′ GCGGCAGGCCTAACACATGCAAG3′; |
| and | |
| Reverse primer: | 5′ ATCCAACCGCAGGTTCCCCTAC3′. |

The 5′ end of the reverse primer was bound via a hexamethylene linker to an amino group in order to attempt binding with a magnetic fine particle. All of the oligonucleotides were synthesized by BEX Co., LTD at our request.

(3) Production of Nucleic Acid Probe Chip

Oligonucleotides 1 and 2 below were synthesized as a probe for detecting (a strand on the reverse primer side of) a PCR amplification product of the above-described E. coli genome and a negative control probe, respectively. The 5′ end of each of the oligonucleotides was bound via a hexamethylene linker to a thiol group.

| | |
|---|---|
| 1: | 5′ CGGACCTCATAAAGTGCGTCGTAGT3′ |
| 2: | 5′ CGTACGATCGATGTAGCTAGCATGC3′ |

A synthetic quartz substrate (size: 25 mm×75 mm×1 mm, manufactured by Iiyama Tokushu Glass) used as a substrate for a nucleic acid probe array was put in a rack resistant to heat and alkali and immersed in a cleaning solution for ultrasonic cleaning adjusted to a predetermined concentration. After being immersed overnight in the cleaning solution, the substrate was ultrasonically cleaned for 20 minutes. The substrate was subsequently taken out of the solution and lightly rinsed with pure water, followed by ultrasonic cleaning in ultrapure water for 20 minutes. Next, the substrate was immersed for 10 minutes in 1 N sodium hydroxide solution heated to 80° C. The substrate was washed again with pure water and ultrapure water to prepare a quartz glass substrate for a probe array.

A silane coupling agent KBM-603 (manufactured by Shin-Etsu Silicones) was dissolved in pure water to bring the concentration to 1%, and stirred at room temperature for 2 hours. Subsequently, the glass substrate previously washed was immersed in the silane coupling agent solution and allowed to stand at room temperature for 20 minutes. After the glass substrate was pulled out of the solution and its surfaces were lightly washed with pure water, the substrate was dried by spraying the both surfaces thereof with nitrogen gas. Next, the dried substrate was baked for 1 hour in an oven heated to 120° C. to complete treatment with the coupling agent, thereby introducing an amino group into the surface of the substrate. Then, A N-(6-Maleimidocaproyloxy) succinimido (hereinafter, abbreviated to EMCS) solution was prepared by dissolving EMCS manufactured by Dojindo Laboratories in a mixed solvent of dimethyl sulfoxide and ethanol (1:1) to bring the final concentration to 0.3 mg/ml. After the completion of baking, the glass substrate was allowed to cool and immersed in the prepared EMCS solution at room temperature for 2 hours. This treatment permitted reaction between the amino group introduced into the surface of the substrate by the silane coupling agent and the succinimide group of the EMCS to introduce a maleimide group into the surface of the glass substrate. The glass substrate pulled out of the EMCS solution was washed using the above-described mixed solvent having the EMCS dissolved therein and subsequently using ethanol, and then dried under nitrogen gas atmosphere.

Each of the above-described probes was dissolved in pure water and dispensed so that the probes each would have the final concentration (when dissolved in a mixed solvent described below) of 10 μM. The resulting solutions were freeze-dried to remove moisture. Next, a solution (mixed solvent) containing 7.5 wt % of glycerin, 7.5 wt % of thiodiglycol, 7.5 wt % of urea and 1.0 wt % of Acetylenol EH (manufactured by Kawaken Fine Chemicals) was prepared. Then, each of the two types of probes previously prepared was dissolved in the above-described mixed solvent to bring the concentration to 10 μM. The obtained DNA solution was filled in an ink tank for a bubble jet printer (trade name: BJF-850, manufactured by Canon), which was in turn installed in a print head.

It is noted that the bubble jet printer used here is a printer modified to be capable of printing on a flat plate. Moreover, this bubble jet printer is adapted to allow the spotting of approximately 10 picoliters of the DNA solution at approximately 120 micrometer pitches by imputing a print pattern according to given file creation procedures.

Using this modified bubble jet printer, a printing operation was performed on one glass substrate to produce a DNA chip. After the printing was confirmed to be reliably performed, the glass substrate was left standing in a humid chamber for 30 minutes, thereby reacting the maleimide group on the surface of the glass substrate with the thiol group at the end of the nucleic acid probe. After 30 minutes, the DNA solution remaining on the surface of the substrate was washed away with 10 mM phosphate buffer (pH 7.0) containing 100 mM NaCl to obtain a DNA chip where the single strand DNA is immobilized on the surface of the glass substrate. The probe for the E. coli genome and the negative control probe each were deposited at 1000 spots on the substrate.

(4) Binding of Magnetic Fine Particle and Primer

Fe particles (diameter: a few nm to 30 nm) were put in a 1 wt % ethanol solution of a silane coupling agent (trade name: KBE-9007, manufactured by Shin-Etsu Chemicals) containing a silane compound having isocyanate groups (3-isocyanatopropyltriethoxysilane), and reacted with stirring at room temperature for 2 hours. Following reaction, the Fe particles were gathered in the lower portion of the reaction solution with a magnet. The Fe particles were then washed three times with ethanol renewed each time, and baked for 10 minutes in an oven heated to 120° C., thereby introducing the isocyanate groups into the magnetic particles.

After the oligonucleotides (10 nm) used as reverse primers were reacted with a portion of the magnetic fine particles treated with the silane coupling agent in 200 μl of 10 mM phosphate buffer (pH 7.0) at room temperature for 2 hours, the primers bound with the magnetic fine particles were gathered using a magnet block for a microtube attached to Genopure (nucleic acid purification kit using magnetic fine particles, manufactured by Bruker Daltonics). After washing operation with pure water was repeated three times, the primers were dissolved (the fine particles were dispersed) in pure water to bring the concentration of the nucleic acid to 10 μM.

(5) PCR Reaction and Strand Separation

Premix PCR reagent (TAKARA ExTaq): 25 μl
Template E. coli genomic DNA: 2 μl (1 ng)
Forward primer: 2 μl (20 pmole)
Reverse primer: 2 μl (20 pmole)
H₂O: 19 μl
(Total: 50 μl)

Three sets of the above-described reaction mixtures were prepared. Using an PCR apparatus (GeneAmp PCR System 9700, manufactured by Applied Biosystems), amplification reaction having a cycle: (1) 95° C., 10 min.→(2) 92° C., 45 sec.→(3) 65° C., 45 sec.→(4) 72° C., 45 sec.→(5) 72° C., 10 min.→(6) 4° C. ((2) to (4) was repeated as cycle reaction) was repeated 3 times, 20 times and 30 times, respectively, for the three sets. The magnetic fine particles were gathered with the magnet block previously described immediately after the amplification product solutions were heated to 90° C. The supernatants were removed, and operation for dispersing the magnetic fine particles in 50 μl of pure water was repeated four times. Ultimately, the magnetic fine particles were dispersed in 120 μl of each buffer for hybridization (6×SSPE).

(6) Hybridization

Three nucleic acid probe chips produced were subjected to hybridization reaction with the 3 sets of the hybridization solutions in a hybridization apparatus (GeneTAC, manufactured by Genomic Solutions). The hybridization was performed at 45° C. for 4 hours, and the nucleic acid probe chips were subsequently washed according to a standard method and finally washed with cold pure water to dry the chips.

(7) Detection by MFM

The nucleic acid probe chips after hybridization were detected using MFM (SPA-400, manufactured by SII NanoTechnology). Result: the number of particles detected at each probe spot (particles/μm: the average of 10 measurements per spot); MFM was conducted at 512 scans/μm in contact mode.
(1) 3 amplification cycles: 0.64
(2) 20 amplification cycles: 11.3
(3) 30 amplification cycles: 978
   This result means that one to plural target nucleic acid molecule(s) bound with one particle is (are) detected and indicates that detection with extremely high sensitivity is made possible.

### Example 2

### Detection without Strand Separation

One of the primers was bound with the magnetic fine particle and subjected to PCR in exactly the same way as Example 1. When detection was conducted without strand separation, the average number of particles detected was as follows:
(1) 3 amplification cycles: 0.65
(2) 20 amplification cycles: 10.7
(3) 30 amplification cycles: 798

This result suggests that a double strand target having a higher concentration when hybridized undergoes the more inhibition of the formation of a hybrid with the probe due to the presence of the complementary strand, and that the amplification of the template having the concentration used does not proceed so much at the completion of the third amplification cycle (although the same holds true for Example 1). As can be seen from the result, the selection of measurement conditions is required for performing detection without practical problems when strand separation is not conducted.

### Example 3

### Detection with Non-Magnetic Fine Particle

A non-magnetic fine particle was used to perform detection in exactly the same way as Example 1 by intentionally using the MFM (which does not enter MFM mode) for comparison. The result has demonstrated that almost the same result as that of Example 1 is obtained as long as 512 scans (in contact mode) are conducted. However, the MFM can conduct rough scans (e.g., 128 scans) in non-contact mode while a scanning speed itself is fast. Thus, the MFM has an advantage that, for example, a method of conducting partially precise scans following rough scans can be adopted.

### Example 4

### Promotion of Reaction by Magnetic Force during Hybridization

Detection was performed in exactly the same way as Example 1 using a hybridization apparatus outlined in FIG. 1 (where reference numeral 3 denotes a magnet). In FIG. 1, reference numeral 1 denotes a temperature controller; reference numeral 2 denotes a nucleic acid probe chip; reference numeral 3 denotes a magnet; reference numeral 4 denotes a nucleic acid probe; and reference numeral 5 denotes a PCR amplification product (single strand) bound with a magnetic fine particle.

When magnetic force was applied to the apparatus for approximately 10 minutes in the early stage of hybridization, almost the same result of detection as that of Example 1 was obtained in 30-minute hybridization. The number of particles that could be detected by merely performing 30-minute hybridization without the application of a magnetic field as in Example 1 was only on the order of 5 to 15% of that in Example 1. Thus, the effect of the application of a magnetic field could be confirmed.

### Example 5

### Separation and Amplification of Only Desired Nucleic Acid from Plural Nucleic Acids

For example, the detection of E. coli in blood will be described in detail. By way of example, the use of an automated nucleic acid purification system (BioRobot EZ1 workstation) and a reagent kit manufactured by Qiagen as an apparatus and a kit for extracting E. coli in blood will be described.

A sample tube, an elution tube, a filter chip and a prepackaged EZ1 reagent cartridge are placed in the apparatus of the automated nucleic acid purification system. Then, purified DNAs are recovered. Both human and bacterial genomes are contained in the recovered nucleic acids. In general, the nucleic acids are directly subjected to PCR amplification with the both of the genomes mixed, and only the desired E. coli DNA was amplified with a primer set. The present Example allows the amplification and detection of only the E. coli DNA by eliminating the human genomic DNA in blood.

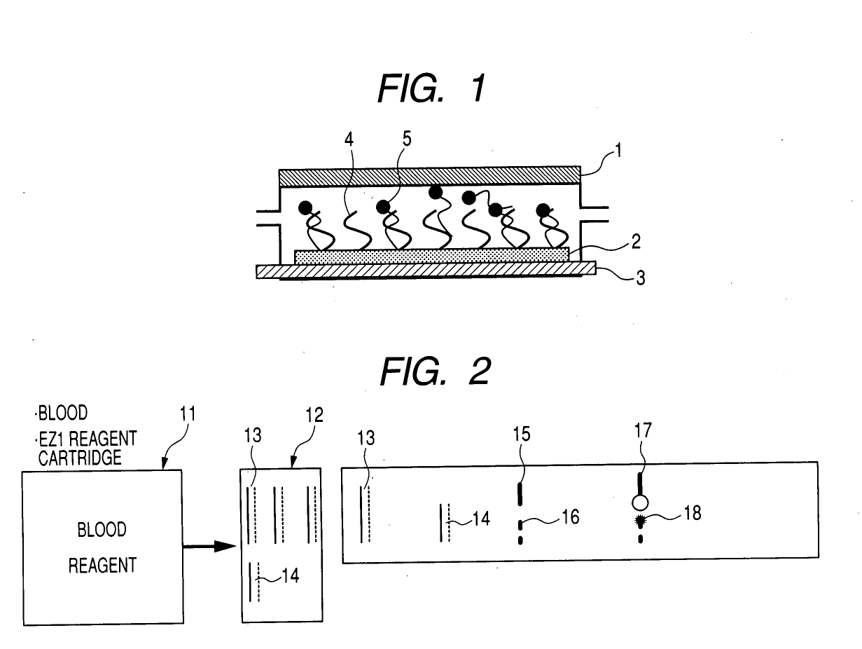

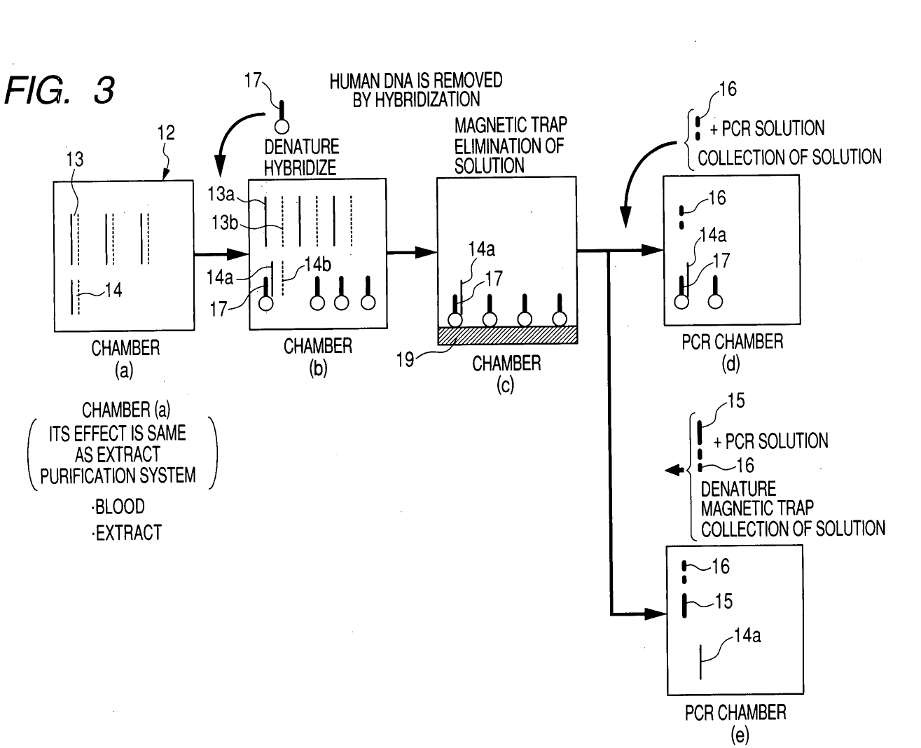

FIG. 2 illustrates preconditions in FIG. 3 and subsequent descriptions. Reference numeral 11 denotes an automated nucleic acid purification system from Qiagen, and Reference numerals 13 and 14 denote human genomic DNA and bacterial DNA, respectively. Reference numeral 12 represents a purified extract where the human genomic DNA 13 and the bacterial DNA 14 are contained. Reference numeral 13a or 13b or reference numeral 14a or 14b in the following figures represents either strand of a double strand DNA composing the human genomic DNA 13 or the bacterial DNA 14, respectively. Reference numeral 15 represents a primer A and reference numeral 16 represents a primer B. The primers A and B are used for amplifying only the DNA of a predetermined region. Reference numeral 17 represents the primer A bound with a magnetic particle and reference numeral 18 represents the primer B bound with a labeling material. FIG. 3 explains procedures for selectively collecting a desired DNA with the magnetic particle primer 17 from the purified extract 12 containing the human genomic DNA 13 and the bacterial DNA 14, followed by the PCR amplification of the collected desired DNA. In (b) of FIG. 3, the purified extract 12 containing the human genomic DNA 13 and the bacterial DNA 14 in (a) of FIG. 3 is supplemented with the magnetic particle primer 17 and heated to a denaturation temperature to separate the double strand of the bacterial DNA 14 into two single strands; after the time sufficiently elapsed, the temperature is allowed to decrease to a temperature at which the magnetic particle primer 17 is hybridized with the bacterial DNA strand 14a; and, after the time sufficiently elapsed, as shown in (c) of FIG. 3., the solution is eliminated from a chamber while the magnetic particle primer 17 is magnetically trapped by a magnet 19. There are two cases of (d) and (e) of FIG. 3. Now referring to (d) of FIG. 3, a first case will be described. A PCR solution and the primer B 16 pairing up with the magnetic particle primer 17 are added to the chamber in (c) of FIG. 3, and the mixture solution is collected and thereby supplied into a PCR chamber, followed by PCR amplification. In a second case shown in (e) of FIG. 3, a PCR solution and a primer set (primer A 15 and prime B 16) are added to the chamber in (c) of FIG. 3, which is in turn heated to a denaturation temperature to subject the hybrid between the magnetic particle primer 17 and the bacterial DNA strand 14a to strand separation. The solution is then collected while the magnetic particle primer 17 is trapped by the magnet 19. The collected solution is supplied into a PCR chamber shown in (e) of FIG. 3 to perform PCR amplification.

Although the use of the automated nucleic acid purification system from Qiagen has been described with reference to FIG. 2, the object of the present invention can also be attained by the procedure shown in (b) of FIG. 3 where a blood sample is added to the nucleic acid extraction reagent and the extracted nucleic acid is hybridized with the magnetic particle primer 17.

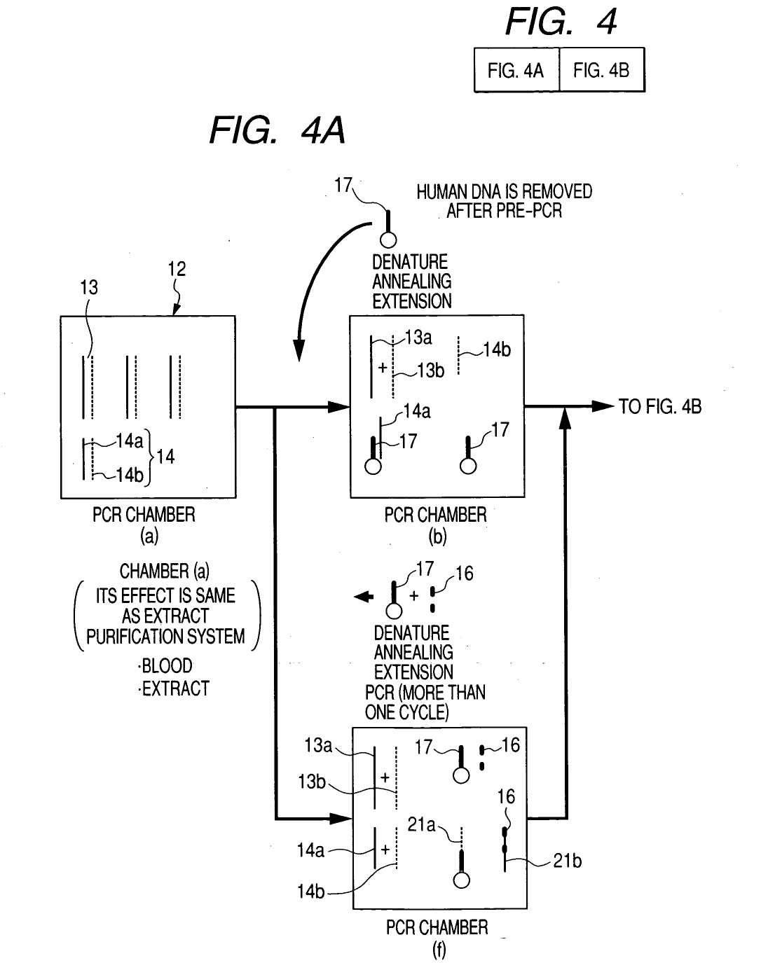

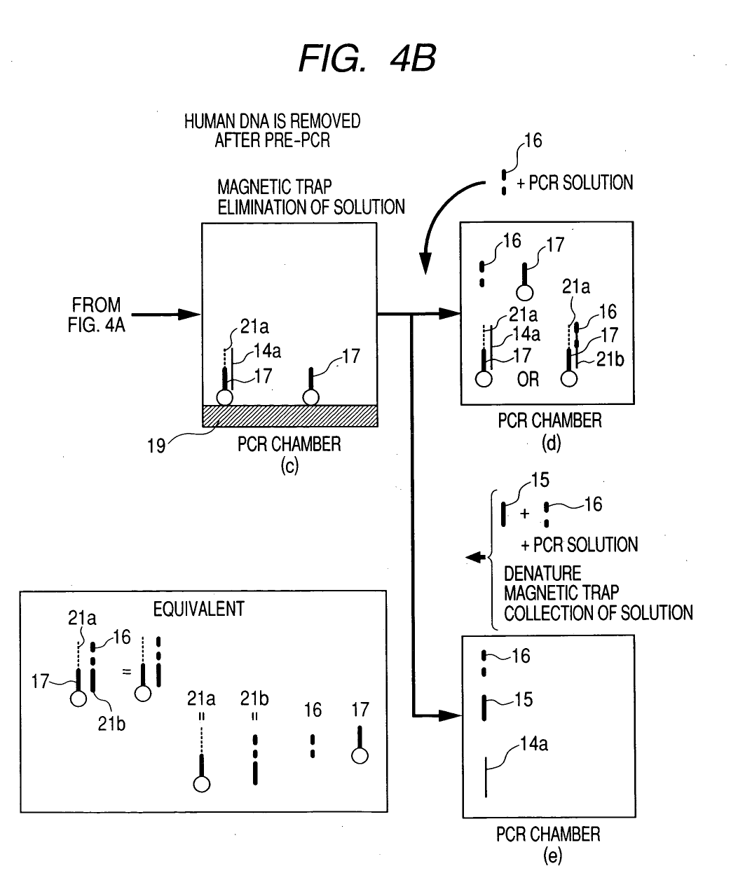

The object of the present invention can be attained even if the division of function between the extraction chamber and the PCR chamber is conducted in any manner. FIG. 4 which consists of FIG. 4A and FIG. 4B illustrates procedures where the mixture containing the human genomic DNA and the bacterial DNA is subjected to pre-PCR amplification once or plural times to collect only the bacterial DNA strand 14a trapped in the magnetic particle primer 17, followed by full-scale PCR amplification using the collected product. (a) of FIG. 4A shows a state in which the human genomic DNA 13 and the bacterial DNA 14 are mixed in the solution in the PCR chamber. In (b) of FIG. 4A, after the magnetic particle primers 17 are added to the solution, the bacterial DNA is separated into single strands at a denaturation temperature and the bacterial DNA strand 14a is annealed to any of the magnetic particle primers 17 at an annealing temperature, followed by base extension at an extension temperature. After the time that allows sufficient base extension, the solution is eliminated while the magnetic particle primers 17 are magnetically trapped by the magnet 19, as shown in (c) of FIG. 4B. The remnants after the solution has been removed are the magnetic particle primer 17, a base extraction product 21a from the magnetic particle primer 17 and the bacterial DNA strand 14a annealed to the magnetic particle primer 17. For performing PCR plural times as shown in (d) of FIG. 4B, a PCR solution and the primer B 16 are added, followed by PCR in plural cycles known in the art. Reference numeral 21b denotes a base extension product from the primer 16. When the process is shifted from (c) to (d) of FIG. 4B, the nucleic acid annealed to the magnetic particle primer 17 is also separated by heating to a denaturation temperature and the solution is eliminated in (c) in FIG. 4B, thereby allowing only the bacterial DNA amplification product 21a to exist as a DNA in (d) of FIG. 4B. In (e) of FIG. 4B, the PCR chamber after the elimination of the solution in (c) of FIG. 4B is supplemented with a PCR solution and heated to a denaturation temperature to separate the bacterial DNA strand 14a that now assumes the form of a double strand annealed to the magnetic particle primer 17 into single strands, followed by the collection of the PCR solution while the magnetic particle primer 17 is trapped by the magnet 19. The collected solution is returned to the PCR chamber from which the magnetic particle primer 17 has been eliminated, to perform PCR in plural cycles. The object of the present invention can be attained even if the primer pair (primer 15 and primer 16) is added at any point of time prior to PCR amplification. In (f) of FIG. 4A, the solution containing the human genomic DNA 13 and the bacterial DNA 14 shown in (a) of FIG. 4A is supplemented with a pair of primers, the magnetic particle primer 17 and the primer 16, to perform PCR in plural cycles. After the bacterial DNA is sufficiently amplified to some degree, procedures similar to the procedures illustrated in (c) of FIG. 4B and subsequent descriptions are performed.

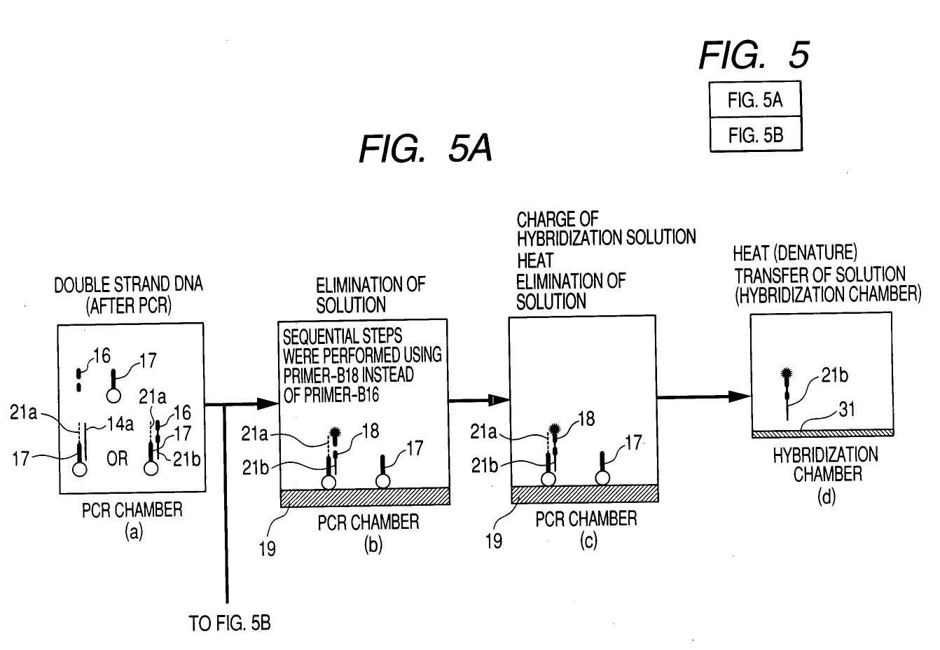

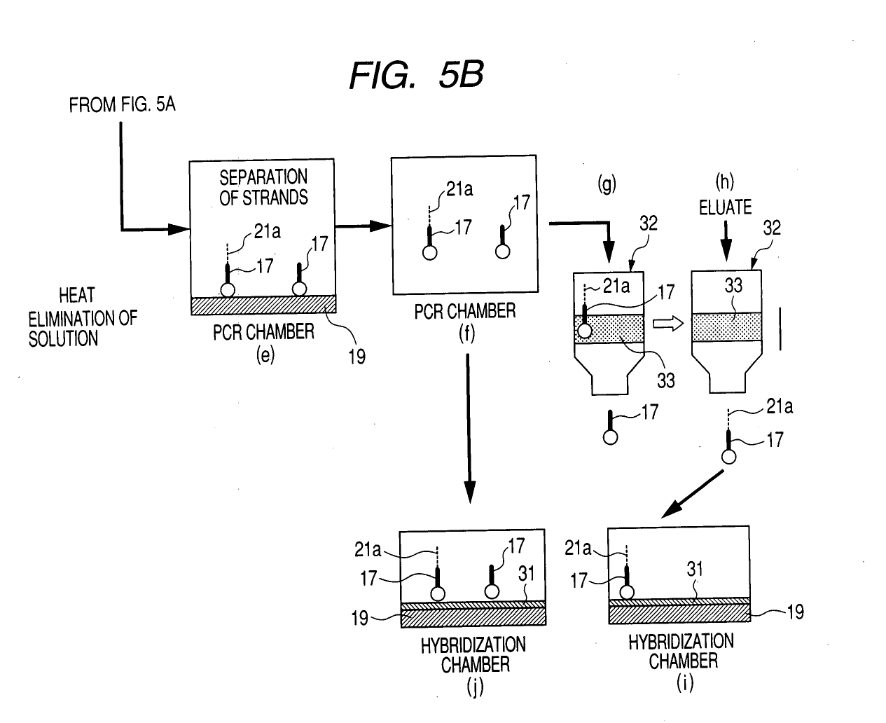

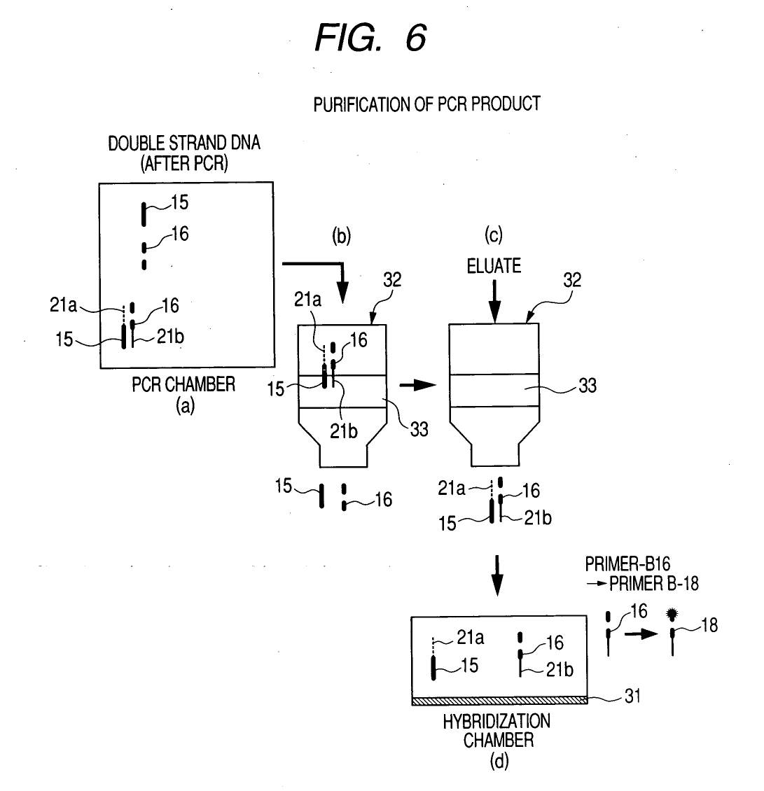

FIGS. 5A and 5B are diagrams for illustrating the detection of the presence or quantity of an amplification product by using an amplification product amplified with a primer pair for PCR amplification (primer 16 and magnetic particle primer 17). FIG. 6 is a diagram for illustrating the detection of the presence or quantity of an amplification product by using an amplification product of the desired bacterial DNA amplified by PCR with a usual primer pair instead of the magnetic particle primer after the human genomic DNA 13 and the bacterial DNA 14 are separated with the magnetic particle primer 17.

In (a) to (b) of FIG. 5A, the PCR solution is eliminated while the magnetic particle primers 17 are magnetically trapped by the magnet 19. As shown in (b) of FIG. 5A, the remnant after the solution has been eliminated is a mixture of the magnetic particle primer 17, the bacterial DNA amplification products 21a and 21b and unreacted magnetic particle primer 17. A hybridization solution is added to the chamber shown in (c) of FIG. 5A which is in turn heated to a denaturation temperature to separate the amplification products 21a and 21b into single strands, followed by the collection of the solution while the magnetic particle primers 17 are magnetically trapped by the magnet 19. Only the amplification product 21b of the bacterial DNA 14 is present in the collected solution. Using this solution, the presence and quantity of the bacterium can be measured by detecting a labeling material after hybridization with a microarray 31. The primer B 18 differs from the primer B 16 in the presence of a labeling material and is a primer formed by the primer 16 bound with a labeling material. Extraction and amplification do not vary depending on the presence of the labeling material. Therefore, although the primer B 18 is used instead of the primer B 16 to explain detection requiring the labeling material, the object and effect of the present invention are not impaired by any means. In order to separate the desired DNA, in (e) of FIG. 5B, a PCR solution containing the human genomic DNA strands 13a and 13b, the bacterial DNA strands 14a and 14b, the bacterial DNA amplification products 21a and 21b and the primers 16 and 17 shown in (a) of FIG. 5A is heated at a denaturation temperature for separating the double strand DNA into single strands. After a lapse of time sufficient for separation into single strands, the PCR solution is eliminated while the magnetic particle primer 17 is magnetically trapped by the magnet 19. As a result, only the extension product 21a of the magnetic particle primer 17 and unreacted magnetic particle primer 17 remain in the PCR chamber as shown in (f) of FIG. 5B. Next, the separation between the magnetic particle primer extension product 21a and the unreacted magnetic particle primer 17 using a PCR purification column 32 will be described. By way of example, the use of QIAquick PCR Purification Kit from Qiagen for the cleanup of DNA from PCR will be described. The QIAquick Kit utilizes a silica gel membrane for the binding of DNA in a high-salt buffer and the elution of DNA with a low-salt buffer or water. By this purification method, primers, nucleotides, enzymes, mineral oil, salts, agarose, ethidium bromide and other impurities can be removed from a DNA sample. The QIAquick silica membrane technology eliminates the problems and inconvenience associated with loose resins and lysates. A binding buffer optimized according to specific applications allows the selective adsorption of DNA fragments with varying sizes. The influence of magnetic particle can be eliminated by reducing its particle diameter. When, for example, a magnetic nanoparticle is used, its influence is significantly reduced. In (g) of FIG. 5B, the magnetic particle primer extension product 21a is trapped in a filter 33 by a high-salt buffer to eliminate other unnecessary substances. Next, as shown in (h) of FIG. 5B, the magnetic particle primer extension product 21a is eluted with a low-salt 15 buffer or water, then supplied into a hybridization chamber, and hybridized with the probe in the microarray 31, followed by detection with the labeling material added to the magnetic particle primer 17 or the labeling material incorporated during nucleic acid extension. Of course, the methods in above-described Examples are applicable as means for detection. As shown in (j) of FIG. 5B, a mixture of the magnetic particle primer extension product 21a and the unreacted magnetic particle primer 17 shown in (f) of FIG. 5B may also be supplied into the hybridization chamber and hybridized with the probe in the microarray 31. After hybridization with the probe array 31, the hybridization solution is eliminated and the microarray 31 is washed, thereby allowing the removal of the magnetic particle primer 17 that is unhybridized.

FIG. 6A shows the PCR solution after PCR that contains a mixture of the amplification products 21a and 21b of the bacterial DNA 14, the primer pair of the primer A 15 and the primer B 16, and so on. FIG. 6B and FIG. 6C show members and effects identical to those in (g) and (h) of FIG. 5B, respectively. The amplification products 21a and 21b are trapped in the filter 33 by passing the solution shown in the FIG. 6A through the filter 33, and the amplification products 21a and 21b trapped in the filter 33 are collected with an eluate and supplied along with the hybridization solution into the hybridization chamber. In FIG. 6D, a double strand pair of the amplification products 21a and 21b is present and is therefore separated into single strands at a denaturation temperature. After sufficient denaturation time, the hybridization solution is placed at a hybridization temperature to hybridize the amplification product 21b with the probe DNA in the microarray 31. Following hybridization, only the amplification product 21b bound with the probe array remains by washing away unnecessary substances with a washing solution. The primer 16 shown in FIG. 6D is changed to the primer 18 attached to the labeling material, and the presence or abundance of the bacterial DNA can be detected by the detection of the labeling material. The change of the primer 16 to the primer 18 attached to the labeling material does not affect extraction and PCR amplification by any means and exhibits, without problems, effect similar to that in the explanation for the above-described procedures.

The present invention allows the convenient and high-sensitivity detection of a nucleic acid, that is, has raised the possibility of opening the new door to gene therapy and so on.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2004-228344 filed on Aug. 4, 2004, which is hereby incorporated by reference herein.

What is claimed is:

1. A detection method of detecting a target nucleic acid using plural nucleic acid probes immobilized on the surface of a substrate, comprising the steps of:
providing a target nucleic acid or a nucleic acid as an amplification reaction product thereof bound with a fine particle;
hybridizing said nucleic acid bound with the fine particle to said immobilized nucleic acid probes; and
detecting said fine particle on said substrate.

2. The detection method of detecting a target nucleic acid according to claim 1,
wherein said step of providing a nucleic acid comprises the steps of:
providing a nucleic acid primer bound with a fine particle; and
subjecting a sample containing the target nucleic acid to strand extension reaction with said primer to obtain a reaction product having an extended strand.

3. The detection method of detecting a target nucleic acid according to claim 2, wherein said step of obtaining a reaction product is the step of performing PCR (polymerase chain reaction).

4. The detection method of detecting a target nucleic acid according to claim 3, wherein said step of obtaining a reaction product is the step of performing PCR using a primer set containing a first primer used as said primer bound with said fine particle and a second primer that is not bound with a fine particle.

5. The detection method of detecting a target nucleic acid according to claim 4, wherein said step of obtaining a reaction product employs plural sets of primers.

6. The detection method of detecting a target nucleic acid according to claim 4, further comprising the step of performing strand separation for separating one single strand DNA having said primer bound with a fine particle as said amplification reaction product, from another single strand DNA by capturing the fine particle of the primer.

7. The detection method of detecting a target nucleic acid according to claim 6, wherein said strand separation is performed by centrifugation.

8. The detection method of detecting a target nucleic acid according to claim 6, wherein said fine particle is a magnetic fine particle, and said strand separation is magnetically performed.

9. The detection method of detecting a target nucleic acid according to claim 4, wherein said first primer is designed for the extension of a strand with a target sequence and wherein a hybrid formed by said hybridization is detected by detecting the fine particle.

10. The detection method of detecting a target nucleic acid according to claim 4, wherein said first primer is designed for the extension of a strand without a target sequence and said second primer is designed for the extension of a strand with a target sequence.

11. The detection method of detecting a target nucleic acid according to claim 2, wherein said primer bound with the fine particle is a primer for linear amplification, and wherein said step of obtaining a reaction product is the step of subjecting a nucleic acid group containing the target nucleic acid to linear amplification reaction with said primer to obtain an amplification reaction product.

12. The detection method of detecting a target nucleic acid according to claim 11, wherein plural primers are simultaneously used.

13. The detection method of detecting a target nucleic acid according to claim 11, wherein said fine particle is a magnetic fine particle.

14. The detection method of detecting a target nucleic acid according to claim 1, wherein said detection is performed using scanning probe microscopy (SPM).

15. The detection method of detecting a target nucleic acid according to claim 14, wherein said SPM is magnetic force microscopy (MFM).

16. A kit for PCR reaction to detect a target nucleic acid, comprising:
plural nucleic acid probes immobilized on the surface of a substrate; and
a primer bound with a fine particle for PCR.

17. The kit for PCR reaction according to claim 16, wherein said fine particle is a magnetic fine particle.
